(19) United States
(12) Patent Application Publication
(10) Pub. No.: US 2003/0220292 A1
(43) Pub. Date: Nov. 27, 2003

(54) Treatment of erectile dysfunction

(75) Inventors: Koichi Okada, Saitama (JP); James L. Yeager, Lake Forest, IL (US); Nadir Buyuktimkin, Robbinsville, NJ (US); Servet Buyuktimkin, Robbinsville, NJ (US)

Correspondence Address: ROGER P. ZIMMERMAN, ESQ., BOWDITCH & DEWEY, LLP, 161 Worcester Road, P.O. Box 9320, Farmingham, MA, 01701-9320, US

(73) Assignee: NexMed (Holdings), Inc., Robbinsville, NJ

(21) Appl. No.: 10/367,109

(22) Filed: Feb. 14, 2003

Related U.S. Application Data
(60) Provisional application No. 60/357,282, filed on Feb. 15, 2002.

(51) Int. Cl. A61K 31/557; A61K 31/736; A61K 9/14

(52) U.S. Cl. 514/54; 514/573; 514/530; 424/487; 424/488

(57) ABSTRACT

Methods for the treatment of erectile dysfunction are provided comprising placing in the fossa navicularis an amount of a semi-solid vasoactive prostaglandin composition sufficient to increase blood flow in the glans penis and resulting in increased tumescence of the penis. In preferred embodiments, the method further comprises providing erotic stimuli. Another embodiment, the invention provides a method for increasing the tumescence of the glans penis. In another aspect, the invention provides compositions and articles of manufacture for the practice of the methods of the invention.

### CROSS REFERENCES TO RELATED APPLICATIONS

The present application claims the benefit of U.S. Provisional Application No. 60/357,282, filed Feb. 15, 2002. The entire contents of the above application are incorporated herein by reference in entirety.

### TECHNICAL FIELD OF THE INVENTION

This invention relates to the compositions and methods for treatment of erectile dysfunction, and more particularly to methods and pharmaceutical compositions for increasing the microcirculation of the glans penis and increasing the tumescence of the glans penis by the administration of medicaments to the fossa navicularis of a patient.

### BACKGROUND OF THE INVENTION

Prior treatments for impotence and erectile dysfunction (ED) have focussed on the task of achieving an erection adequate for intercourse, and specifically for vaginal penetration. While progress has been made in this area, there remains the problem that an erection that is adequate may not be satisfactory to either the patient or his sexual partner.

The term “impotence” has been used to signify the inability of the male to attain and maintain erection of the penis sufficient to permit satisfactory sexual intercourse. The term “erectile dysfunction” has been suggested as a more precise term “to signify an inability of the male to achieve an erect penis as part of the overall multifaceted process of male sexual function.” Droller, M. J. et al. Impotence. Consensus Development Conference Statement, National Institutes of Health (1993).

Erectile dysfunction may result from psychological causes (psychogenic erectile dysfunction) or organic causes or a combination of both. Organic causes include physiological, nervous, vascular and hormonal pathologies or a combination thereof.

The normal physiology of an erection involves nerve impulses that signal certain muscles to relax. These muscles, when contracted, restrict blood flow through arteries in the penis. When relaxed, the muscles permit a significant increase in blood flow. The increased blood flow engorges three groups of erectile tissue within the penis with blood and the penis becomes less flaccid. The engorged erectile tissue and the muscle structure of the penis depress adjacent veins, restricting the flow of blood out of the penis. The restriction of blood flow out of the penis increases and sustains the erection.

Deficiencies of some hormones, such as testosterone, or elevation of others, such as prolactin, can cause erectile dysfunction. Many drugs, such diuretics, antihypertensives, anticonvulsants, narcotics, alcohol, and psychotropic drugs may cause erectile dysfunction as a side effect. Murray, F. T. et al. Amer. J. Medical Sci. 309: 99-109 (1995).

Damage to nerves and blood vessels may also provide an organic cause for erectile dysfunction. Disease processes may involve several aspects. For example, diabetes, which causes damage to both nerves and blood vessels, can cause erectile dysfunction. A significant percent of all diabetic men will suffer from erectile dysfunction.

Methods proposed for the treatment of erectile dysfunction have included external devices, sex therapy, surgical implantation of internal prostheses, injection of drugs directly into the penis and topically applied medications. None of these approaches is entirely effective.

External devices include tourniquets (see U.S. Pat. No. 2,818,855) and externally applied vacuum erection aids. While some clinicians consider externally applied erection aids as a first option for treatment, some patients are unwilling to use such devices. O'Keefe, M., et al. Medical Clinics of North America 79: 415-434 (1995).

Symptomatic sex therapy was originally found to be effective by Masters and Johnson, but later studies have not shown as impressive results. Freudian therapy does not appear to patients to be an attractive alternative. Vickers, M. A., et al. J. Urology 149: 1258-1261 (1993).

Surgically implanted mechanical devices, such as hinged or solid rods and inflatable, spring driven or hydraulic prostheses have been used for some time. Several penile prostheses have been described that are pliable plastic elements with constant rigidity. However, these prostheses are continuously rigid and can cause discomfort for the patient. Other types of penile prostheses include surgically positioned pumps for creating high pressure of liquid in the elastic silicon mantle. Implantation of these complex devices requires implanting several components into the patient's body, for example, the reservoir with liquid, pump, several valves, connecting pipes, and the like, in addition to those components implanted directly into the penis. Other penile prostheses use an external source of electricity and a source of an alternating magnetic field changing with the frequency of 50 to 1000 Hz that influences the internal element located in the penis. This element senses the magnetic field and causes liquid in the inner reservoir to move from the reservoir into the elastic mantles located in the corpora cavernosa, and causes the penis to erect. Some disclosures describe prostheses including a permanent magnet that makes seesaw movements under the influence of this field, which in turn causes liquid from the reservoir to pump into the elastic mantles, thus serving as an internal element sensing alternating magnetic field of the external source. However, while such prostheses can provide adequate rigidity for intercourse, patients and the patients' partners have been reported to indicate unmet expectations with their penile prostheses. Case reports have recounted the results of treating a single patient with intracavernosal injections of PGE₁ (Keogh, E. J. and Earle, C. M., Int. J. Impotence Res., 4:113, 1992) or with MUSE® (Chew, K. K., & Stuckey, B. G. A., Int. J. Impotence Res., 12: 195-196, 2000) in an attempt to alleviate dissatisfaction with a penile prosthesis.

The administration of erection effecting and enhancing drugs is taught in U.S. Pat. No. 4,127,118 to LaTorre. This patent teaches a method of treating male impotence by injecting into the penis an appropriate vasodilator, in particular, an adrenergic blocking agent or a smooth muscle relaxant to effect and enhance an erection.

More recently, U.S. Pat. No. 4,801,587, to Voss et al. teaches the application of an ointment to relieve impotence. The ointment consists of the vasodilators papaverine, hydralazine, sodium nitroprusside, phenoxybenzamine, or phentolamine and a carrier to assist absorption of the primary agent through the skin. U.S. Pat. No. 5,256,652 to El-Rashidy teaches the use of an aqueous topical composition of a vasodilator such as papaverine together with hydroxypropyl-β-cyclodextrin.

Prostaglandin E₁ is a derivative of prostanoic acid, a 20-carbon atom lipid acid, represented by the formula:

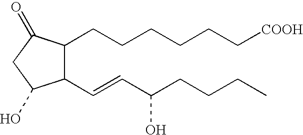

and is commercially available, e.g., from Chinoin Pharmaceutical and Chemical Works Ltd. (Budapest, Hungary) under the designation “Alprostadil USP,” from Pharmacia & Upjohn under the designation “Caverject”. Prostaglandin E₁ complexed with alpha-cyclodextrin is available as alprostatil alfadex from Ono Pharmaceuticals (Japan) and in an injectable form under the designation “Edex®” or “Viradex®” from Schwarz Pharma (Germany).

In one commercially available form (MUSE®, Vivus, Menlo Park Calif.), alprostadil is administered in a pellet deposited in the urethra using an applicator with a hollow stem 3.2 cm in length and 3.5 mm in diameter (Padma-Nathan, H., et al., N. Engl. J. Med., 336: 1-7 (1997), see especially FIG. 1). In the home treatment portion of the Padma-Nathan et al. study, 32.7% of the patients (10.8% of administrations) receiving MUSE® complained of penile pain and 5.1% experienced minor urethral trauma, compared to 3.3% and 1.0%, respectively, of the patients receiving placebo. Frequency of report of these side effects has varied in subsequent studies: MUSE® producing penile pain in 17-23.6% of administrations, compared to 1.7% with placebo and minor urethral bleeding reported by 4.8% of patients (Peterson, C. A., et al., J. Urol., 159: 1523-1528 (1998)). In a study on a European population, 31% MUSE® patients reporting penile pain or burning sensations, 4.8% reporting urethral bleeding, and 2.9% reporting severe testicular pain (Porst, H., Int. J. Impot. Res., 9:187-192 (1997)). The percent of patients responding to MUSE® treatment, defined as having at least one erection considered sufficient for intercourse, has been reported to be 43% (Porst, 1997), 65.9% (Padma-Nathan et al., 1997) and 70.5% (Peterson et al., 1998), although published editorial comment has suggested that the percent of patients responding in the latter two studies is more properly reported as 30-40% (Benson, G., J. Urol., 159: 1527-1528 (1998).

Intraurethral application of a preparation of 1 mg prostaglandin E₁ in phosphatidylcholine liposomes in 1 ml polyoxyethylene glycol has been reported to be less effective than intracavernosal injection of prostaglandin E₁ (Englehardt, P. F., et al., British J. Urology, 81: 441-444, 1998). No ED patients receiving the liposomal preparation achieved complete penile rigidity, and only 6 of 25 patients achieved an erection adequate for vaginal penetration. In contrast, intracavernosal injection of prostaglandin E₁ produced erections adequate for vaginal penetration or complete rigidity in 23 of 25 of the same ED patients. The authors suggested that the transurethral effect of the prostaglandin E₁ probably arises by diffusion of prostaglandin E₁ first into the corpus spongiosum and then into the corpus cavernosum.

While the above mechanical and pharmaceutical treatments have focussed on producing adequate penile rigidity, even when the treatments succeed in producing adequate rigidity, the satisfaction of the patient and the patient's sexual partner is often less than adequate. Patients discontinue medical treatments that produce rigidity, such as intracavenosal injections or transurethral suppositories because of painful side effects. Penile implants may produce rigidity, but insufficient tumescence. In particular, lack of tumescence of the glans penis is a recognized source of dissatisfaction for both the patient and the sexual partner (See, e.g., U.S. Pat. No. 6,418,934; Chew & Stuckey, 2000).

### SUMMARY OF THE INVENTION

The present invention provides methods for the treatment of erectile dysfunction comprising placing a semi-solid composition comprising an effective amount of a vasoactive prostaglandin in the fossa navicularis and providing erotic stimulation resulting in an increase in blood flow in the glans and an increased intumescence of the penis. In preferred embodiments, the treatment results in intumescence of the glans, and preferably in the presence of erotic stimuli, a penile erection adequate for intercourse. At least one erotic stimulus is selected from the group consisting of olfactory stimuli, visual stimuli, auditory stimuli and tactile stimuli. In a preferred embodiment, the erotic stimuli are provided by the patient or the patient's sexual partner.

In one embodiment, the invention provides a method of treating erectile dysfunction in a patient needing such treatment comprising the steps of:

placing in the fossa navicularis of the patient an amount of a semi-solid vasoactive prostaglandin composition effective to increase blood microcirculation in the glans penis, comprising a dose of about 0.05 mg to about 0.8 mg of a vasoactive prostaglandin, a penetration enhancer, a polymer selected from the group consisting of polysaccharide gums and polyacrylic acid polymers, a lipophilic component that is selected from the group consisting of an aliphatic C₁ to C₈ alcohol, an aliphatic C₈ to C₃₀ ester, and a mixture thereof; and an acidic buffer system; and

providing at least one erotic stimulus selected from the group consisting of olfactory stimuli, visual stimuli, auditory stimuli and tactile stimuli. In a preferred embodiment, the vasoactive prostaglandin is prostaglandin E₁. Suitably, the dose of the prostaglandin E₁ is about 0.05 mg to about 0.8 mg, preferably about 0.1 mg to about 0.5 mg. In another embodiment, the dose of the prostaglandin E₁ is about 0.2 mg to about 0.3 mg.

In one embodiment, the invention provides a method of enhancing tumescence of the glans penis comprising the step of placing in the fossa navicularis of the patient an amount of a semi-solid vasoactive prostaglandin composition sufficient to increase blood flow in the glans penis. In a preferred embodiment, the method further comprised providing an amount of the composition effective to produce tumescence of the corpora cavernosa.

The semi-solid vasoactive prostaglandin composition suitable for the practice of the method of the present invention comprises a vasoactive prostaglandin, preferably prostaglandin E₁, a penetration enhancer, a polymer selected from the group consisting of a polysaccharide gum and a polyacrylic acid polymer, a lipophilic component, and an acidic buffer system. In a preferred embodiment, the penetration enhancer is an alkyl-2-(N-substituted amino)-alkanoate ester, an (N-substituted amino)-alkanol alkanoate, or a mixture of these. Typically, the lipophilic component is selected from the group consisting of an aliphatic C₁ to C₈ alcohol, an aliphatic C₂ to C₃₀ ester, aliphatic C₈ to C₃₀ ester or a mixture of these. The composition includes a buffer system that provides a buffered pH value for the composition in the range of about 3 to about 7.4. A preferred pH value is about 3.0 to about 7.4, more preferably about 3.0 to about 6.5, most preferably from about 3.5 to about 6.0. If desired, stabilizers, preservatives and emulsifiers may be included. In some embodiments, the composition exhibits non-Newtonian rheological properties, suitably comprising a shear-thinning polysaccharide gum or a shear-thinning polyacrylic acid polymer. In one embodiment, the composition is thixotropic. In another embodiment, the composition is pseudoplastic.

In one embodiment the composition comprises, in addition, an effective dose of a piperazinyl quinazoline antihypertensive. Suitable piperazinyl quinazolines include alfuzosin, bunazosin, doxazosin, prazosin, terazosin, trimazosin and mixtures thereof.

In another aspect, the invention provides a unit dose an article of manufacture comprising container of a composition comprising about 0.05 mg to about 0.8 mg of a vasoactive prostaglandin and a penetration enhancer, and labeling instructions.

Other and further aims, purposes, features, advantages, embodiments and the like will be apparent to those skilled in the art from the present specification and the appended claims.

### BRIEF DESCRIPTION OF THE DRAWINGS

FIG. 1 is a diagram of the anatomical structure of the human penis in longitudinal section view;

FIG. 2 is a diagram of the anatomical details of the distal portion of the human penis in longitudinal section view;

FIG. 3 is a graphical representation of the results of a laser doppler flowmeter measurement of the blood flow in the glans penis of a 65 year old patient not exhibiting erectile dysfunction (IIEF-5 score: 24, BP 137/82) before, during and after treatment using the method of the present invention with a semisolid composition comprising 0.3 mg prostaglandin E₁ at the time indicated by the arrow, showing an increase in blood flow following the treatment (left arrow) followed by an erection at 14 minutes (right arrow);

FIG. 4 is a graphical representation of the results of a laser doppler flowmeter measurement of the blood flow in the glans penis of a 51 year old erectile dysfunction patient (IIEF-5 score: 10, severe ED) before, during and after treatment using the method of the present invention with a semisolid composition comprising 0.2 mg prostaglandin E₁ at the time indicated by the arrow, showing an increase in blood flow following the treatment, with the glans reaching tumescence in 9 minutes;

FIG. 5 is a graphical representation of the results of a laser doppler flowmeter measurement of the blood flow in the glans penis of a 57 year old erectile dysfunction patient (IIEF-5 score: 15, moderate ED, BP 124/50) before, during and after treatment using the method of the present invention with a semisolid composition comprising 0.2 mg prostaglandin E₁ at the time indicated by the arrow, showing an increase in blood flow following the treatment, with the glans reaching tumescence in 4 minutes; and

FIG. 6 is a graphical representation of the results of a laser doppler flowmeter measurement of the blood flow in the glans penis of a 56 year old erectile dysfunction patient (IIEF-5 score: 9, severe ED, BP 144/88) before, during and after treatment using the method of the present invention with a semisolid composition comprising 0.2 mg prostaglandin E₁ at the time indicated by the left arrow, showing an increase in blood flow following the treatment, with the glans reaching tumescence in 7 minutes (right arrow).

FIGS. 7A and 7B are graphical representations of the results of a laser doppler flowmeter measurement of the blood flow in the glans penis of a 60 year-old patient suffering from erectile dysfunction (IIEF-5 score: 14, moderate ED, BP 145/88) before, during and after treatment using the method of the present invention with a semisolid composition comprising 0.2 mg prostaglandin E₁. FIG. 7A: 0.2 mg prostaglandin E₁ was administered in the absence of sexual stimuli (“SS(−)”) at the time indicated by the arrow, showing an increase in blood flow following the treatment, with the glans reaching tumescence in about 6 minutes (arrow). Glans blood flood increased to the level expected during a physiologically normal erection within ten minutes, but no erection developed under the conditions and surroundings of the study in the absence of sexual stimulation. In FIG. 7B, the same dose of prostaglandin administered in the presence of audio-visual erotic stimuli (“AVSS(+)”) produced a larger and more rapid increase in glans blood flow and tumescence. A rigid erection was obtained that was maintained for more than one hour.

### DETAILED DESCRIPTION OF THE INVENTION

It has been found that that a semi-solid prostaglandin E₁ composition suitable for the treatment of erectile dysfunction can be placed advantageously in a natural enlarged space immediately proximal to the penile meatus, the fossa navicularis, resulting in an increase in blood flow in the glans penis and an increased tumescence of the penis. In a preferred embodiment, the treatment of erectile dysfunction patients using the method and composition of the present invention in combination with erotic stimuli results in increased intumescence of the glans penis as well as a penile erection sufficient for intercourse.

The fossa navicularis provides a restricted site that is ideally suited for the application of pharmaceutical compositions. The space is lined bm a non-keratinized stratified squamous epithelium and is thereby distinguished from the surface skin covering the glans and the rest of the penis and from the stratified columnar epithelium of the lining of the urethra proper. It has been found that the administration of the composition of the present invention in the fossa navicularis has unexpectedly high efficacy and low incidence of local side effects.

The fossa navicularis provides a natural space adaptable to the application and retention of pharmaceutical compositions. A semi-solid medicament when placed in the fossa has higher impedance to flow at narrowed exits of this space, the meatus and the urethra. Thus, a semi-solid medication of suitably chosen viscosity is naturally retained within the fossa, facilitating the absorption of active agents such as vasodilators.

The fossa navicularis is part of the natural defense system that protects the body against infection. The fossa navicularis is a more immunologically protected site than the adjacent pars spongiosa region of the penile urethra proper. Depositing a semisolid medicament within the anatomical limits of the fossa navicularis thus does not circumvent the natural barriers to disease by artificially transporting contaminants, e.g., from the surface of the penis, directly into the penile urethra proper. As noted above, the fossa navicularis naturally supports a bacterial flora that maintains an acid pH.

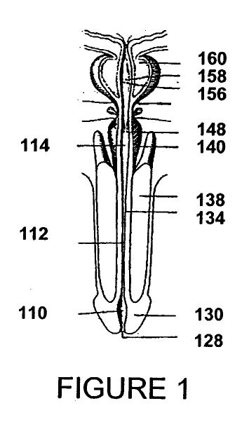

Referring to FIG. 1, the basic structure of the human penis is illustrated. The fossa navicularis 110 is a natural enlargement of the lumen of the male urethra that extends distally to the urethral meatus (penile meatus or “ostium”) 128 and proximally to the pendulous region of the urethra 112 (also termed “pars spongiosa” region of the urethra), the portion of the urethra that passes through the corpus spongiosum 134. The bulbar urethra 114 is proximal to the pendulous region of the urethra, and passes through the bulbospongiosus muscle 140. More proximally, the opening 148 in the wall of the urethra of the bulbourethral glands (Cowper's glands) can be seen. More proximally, the urethra passes through the prostate gland 160, where openings ejaculatory duct 156 and of the prostate utricle 158 are visible in the wall of the urethra. Engorgement with blood of erectile tissues of the glans penis 130, corpus spongiosum 134 and corpora cavernosa 138 produces an erection of the penis.

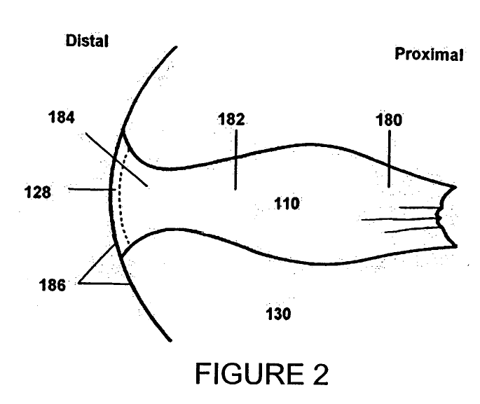

Referring to FIG. 2, the detailed structure of the fossa navicularis 110 is illustrated. The external opening, the meatus 128, is the distal limit of the fossa navicularis. The external skin of the glans is covered by a keratinized stratified squamous epithelium 186 (Pudney, J., and Anderson, D. J., (1995) Immunobiology of the human penile urethra, Amer. J. Path., 147: 155-165) that is marked by proximally by a sharp transition (dashed line) to the nonkeratinized stratified squamous epithelium without glycogen 184 that is characteristic of the lining of the distal fossa navicularis.

The fossa navicularis widens proximally and the lining changes to a nonkeratinized stratified squamous epithelium with glycogen 182. The glycogen in this region is believed to support a bacterial flora that lowers the pH of the region and contributes to a natural defense against infection. Holstein, A. F., et al., (1991) Different epithelia in the distal human male urethra, Cell Tiss. Res. 264: 23-32. This nonkeratinized stratified squamous epithelium with glycogen is under hormonal control, and increases in extent under increased estrogen levels. (Holstein, et al., 1991. The proximal fossa navicularis narrows in width, and is lined by a stratified columnar epithelium 180.

Semi-solid compositions and penetration enhancers suitable for the practice of the present invention are described in detail in U.S. Pat. Nos. 6,046,244, 6,118,020 and 6,323,241, the teachings of which are incorporated herein by reference.

The semi-solid composition has a suitably chosen viscosity such that the composition is naturally retained within the fossa navicularis. The semi-solid composition can exhibit Newtonian or non-Newtonian Theological characteristics. In some preferred embodiments, the semi-solid composition of the present invention exhibits non-Newtonian Theological characteristics, i.e. in which the apparent viscosity is dependent on the shear rate applied to the composition. Preferably the composition has “shear-thinning” rheological properties. As used herein, “shear-thinning” refers to a reduction in apparent viscosity (the ratio of shear stress to the shear rate) with increasing shear rate, whether the reduction in apparent viscosity is time independent (pseudoplastic), time dependent (thixotropic) or associated with a yield stress, defined as a stress that must be exceeded before flow starts, (Bingham plastics and generalized Bingham plastics). See, generally, Harris, J., & Wilkinson, W. L., “Non-newtonian Fluid,” pp.856-858 in Parker, S. P., ed., McGraw-Hill Encyclopedia of Physics, Second Edition, McGraw-Hill, New York, 1993.

In a preferred embodiment, the pharmaceutical composition comprises at least one vasoactive prostaglandin, preferably prostaglandin E₁, an alkyl (N-substituted amino) ester, a polysaccharide gum, a lipophilic component, and an acid buffer system.

Vasoactive prostaglandins are those that act as peripheral vasodilators, including naturally occurring prostaglandins such as PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}; semisynthetic or synthetic derivatives of natural prostaglandins, including carboprost tromethamine, dinoprost tromethamine, dinoprostone, lipoprost, gemeprost, metenoprost, sulprostone and tiaprost. Prostaglandin and prostaglandin E₂ are particularly preferred vasoactive prostaglandins for use in conjunction with the present method.

Additionally, simultaneous administration of one or more non-ecosanoid vasodilators may be desirable and may in some cases exhibit a synergistic effect. The combination of prazosin with prostaglandin E₁ has been found to be particularly advantageous in this regard; the latter drug appears to act as a potentiator for prazosin.

Suitable non-ecosanoid vasodilators include, but are not limited to: nitrates such as nitroglycerin, isosorbide dinitrate, erythrityl tetranitrate, amyl nitrate, sodium nitroprusside, molsidomine, linsidomine chlorhydrate (“SIN-1”) and S-nitroso-N-acetyl-d,l-penicillamine (“SNAP”); amino acids such as L-arginine; long and short acting α-adrenergic blockers such as phenoxybenzamine, dibenamine, phentolamine, tamsulosin and indoramin, especially quinazoline derivatives such as alfuzosin, bunazosin, doxazosin, terazosin, prazosin, and trimazosin; vasodilative natural herbal compositions and bioactive extracts thereof, such as gosyajinki-gan, Satureja obovata, bai-hua qian-hu, lipotab, saiboku-to, vinpocetine, Gingko biloba, bacopa, Gynostemma pentaphyllum, gypenosides, Evodia rutaecarpa, rutaecarpine, dehydroevodiamine, dan-shen, salviae miltiorrhizae radix, shosaikoto, Zizyphi fructus, ginseng and mixtures thereof (U.S. Pat. No. 6,007,824); ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; vasodilators such as nimodepine, pinacidil, cyclandelate, dipyridamole and isoxsuprine; chlorpromazine; haloperidol; yohimbine; trazodone and vasoactive intestinal peptides.

Prostaglandin E₁ is well known to those skilled in the art. Reference may be had to various literature references for its pharmacological activities, side effects, and normal dosage ranges. See for example, Physician's Desk Reference, 51st Ed. (1997), The Merck Index, 12th Ed., Merck & Co., N.J. (1996), and Martindale The Extra Pharmacopoeia, 28th Ed., London, The Pharmaceutical Press (1982). Prostaglandin E₁ as well as other compounds referenced herein are intended to encompass pharmaceutically acceptable derivatives including physiologically compatible salts and ester derivatives thereof.

The quantity of vasoactive prostaglandin, such as prostaglandin, in the pharmaceutical composition is a therapeutically effective amount and necessarily varies according to the desired dose, the dosage form (e.g., suppository or topical), and the particular form of vasoactive prostaglandin used. The term “prostaglandin” as used generically herein refers to the prostaglandin free acid and pharmaceutically acceptable derivatives thereof, including, for example PGE₁, pharmaceutically acceptable salts and lower alkyl esters thereof (the term “lower alkyl” as used herein means straight chain or branched chain alkyl containing one to four carbon atoms). The composition generally contains between 0.001 percent to 1 percent of vasoactive prostaglandin, e.g., prostaglandin E₁, typically contains between 0.05 percent to 1 percent, preferably from 0.1 percent to 0.5 percent, based on the total weight of the composition.

When used in combination with a vasoactive prostaglandin, a piperazinyl quinazoline antihypertensive, such as prazosin, is present in the amount of about 0.1 mg to about 2.0 mg per unit dose, depending on the potency of the particular piperazinyl quinazoline antihypertensive and the type and dose of vasoactive prostaglandin used. The dose and the proportion of vasoactive prostaglandin and the piperazinyl quinazoline antihypertensive can be routinely determined by one of ordinary skill without undo experimentation.

Working alone, most drugs, prostaglandin formulations included, do not sufficiently permeate the skin to provide drug concentration levels comparable to those obtained from other drug delivery routes. To overcome this problem, topical drug formulations typically include a skin penetration enhancer. Skin penetration enhancers also may be referred to as absorption enhancers, accelerants, adjuvants, solubilizers, sorption promoters, etc. Whatever the name, such agents serve to improve drug absorption across the skin. Ideal penetration enhancers not only increase drug flux across the skin, but do so without irritating, sensitizing, or damaging skin. Furthermore, ideal penetration enhancers should not adversely affect the physical qualities of the available dosage forms (e.g. cream or gel), or the cosmetic quality of the topical composition.

A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Büyüktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997).

Suitable penetration enhancers for use in prostaglandin topical compositions are disclosed in U.S. Pat. No. 4,980,378, 5,082,866 and 6,118,020. Topical compositions employing such penetration enhancers for the delivery of prostaglandins are disclosed in U.S. Pat. Nos. 6,046,244, 6,323,241, 6,414,028, and 6,489,207.

The topical composition of the present invention can contain one or more penetration enhancers. Among the preferred penetration enhancers for the present invention are ethanol, propylene glycol, glycerol, ethyl laurate, isopropyl palmitate, isopropyl myristate, laurocapram (Azone™), dioxolanes (described in U.S. Pat. No. 4,861,764), macrocyclic ketones, HP-101, oxazolidones and biodegradable penetration enhancers (described in U.S. Pat. Nos. 4,980,378 and 5,082,866 to Wong et al. such as alkyl-2-(N,N-disubstituted amino) alkanoates (e.g., dodecyl N,N-dimethylamino isoproprionate (DDAIP)), N,N-disubstituted amino alkanol alkanoates) and mixtures thereof. The penetration enhancer is present in an amount sufficient to enhance the penetration of the vasoactive prostaglandin, e.g., prostaglandin E₁. The specific amount varies necessarily according to the desired release rate and the specific form of prostaglandin E₁ used. Generally, the penetration enhancer is present in an amount ranging from about 0.5 weight percent to about 20 weight percent, based on the total weight of the composition. Preferably, the penetration enhancer is present in an amount ranging from about 1 weight percent to about 10 weight percent of the composition. More preferably, the penetration enhancer is present in an amount ranging from about 1 weight percent to about 5 weight percent of the composition.

In general, suitable penetration enhancers can be chosen from those listed above as well as sulfoxides, alcohols, fatty acids, fatty acid esters, polyols, amides, surfactants, terpenes, alkanones, organic acids and mixtures thereof. See generally Chattaraj, S. C. and Walker, R. B., Penetration Enhancer Classification, pp.5-20 in Maibach, H. I., and Smith, H. E., (eds.), Percutaneous Penetration Enhancers, CRC Press, Inc., Boca Raton, Fla. (1995) and Büyüktimkin, N., et al., Chemical Means of Transdermal Drug Permeation Enhancement, in Gosh, T. K., et al., (eds.) Transdermal and Topical Drug Delivery Systems, Interpharm Press, Inc., Buffalo Grove, Ill. (1997). Suitable sulfoxides include dimethylsulfoxide, decylmethylsulfoxide and mixtures thereof. Suitable alcohols include ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, olcyl alcohol, linolyl alcohol, linolenyl alcohol and mixtures thereof. Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol and mixtures thereof.

Suitable amides include urea, dimethylacetamide, diethyltoluamide, dimethylformamide, dimethyloctamide, dimethyldecamide, 1-alkyl-4-imidazolin-2-one, pyrrolidone derivatives, cyclic amides, hexamethylenelauramide and its derivatives, diethanolamine, triethanolamine and mixtures thereof. Suitable pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-decyl-thioethyl-2-pyrrolidone (HP-101), 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkypyrrolidone, fatty acid esters of N-(2-hydroxymethyl)-2-pyrrolidone and mixtures thereof. Suitable cyclic amides include 1-dodecylazacycloheptane-2-one (laurocapram, Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexane-2-one, 1-geranylazacyclopentan-2,5-dione, 1-farnesylazacyclopentan-2-one and mixtures thereof.

Suitable surfactants include anionic surfactants, cationic surfactants, nonionic surfactants, bile salts and lecithin. Suitable anionic surfactants include sodium laurate, sodium lauryl sulfate and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxy-poly(oxyethylene)-poly(oxypropyl) poly(oxyethylene)block copolymers, polyoxyethylene ethers, polyoxyethylene sorbitan esters, polyethylene glycol esters of fatty alcohols and mixtures thereof. Suitable α-hydro-ω-hydroxy-poly(oxyethylene)-poly(oxypropyl) poly(oxyethylene)block copolymers include Poloxamers 231, 182, and 184 and mixtures thereof. Suitable polyoxyethylene ethers include 4-lauryl ether (Brij 30), (Brij 93), (Brij 96), 20-oleyl ether (Brij 99) and mixtures thereof. Suitable polyoxyethylene sorbitan esters include the monolaurate (Tween 20, Span 20) the monopalmitate (Tween 40), the monostearate (Tween 60), and the monooleate (Tween 80) and mixtures thereof. Suitable polyethylene glycol esters of fatty acids include the 8-oxyethylene stearate ester (Myrj 45), (Myrj 51), the 40-oxyethylene stearate ester (Myrj 52) and mixtures thereof. Suitable bile salts include sodium cholate, sodium salts of laurocholic, glycolic and desoxycholic acids and mixtures thereof.

Suitable terpenes include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineole, ylang ylang oil, anise oil, chenopodium oil, eucalyptus oil and mixtures thereof Suitable alkanones include N-heptane, N-octane, N-nonane, N-decane, N-undecane, N-dodecane, N-tridecane, N-tetradecane, N-hexadecane and mixtures thereof. Suitable organic acids include citric acid, succinic acid, salicylic acid, salicylates (including the methyl, ethyl and propyl glycol derivatives), tartaric acid and mixtures thereof.

In a preferred embodiment, the penetration enhancer is an alkyl-2-(N-substituted amino)-alkanoate, an (N-substituted amino)-alkanol alkanoate, or a mixture of these. For convenient reference, alkyl-2-(N-substituted amino)-alkanoates and (N-substituted amino)-alkanol alkanoates can be grouped together under the label alkyl (N-substituted amino) esters.

Alkyl-2-(N-substituted amino)-alkanoates suitable for the present invention can be represented as follows:

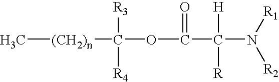

wherein n is an integer having a value in the range of about 4 to about 18; R is a selected from the group consisting of hydrogen, C₁ to C₇ alkyl, benzyl and phenyl; R₁ and R₂ are selected from the group consisting of hydrogen and C₁ to C₇ alkyl; and R₃ and R₄ are selected from the group consisting of hydrogen, methyl and ethyl.

Preferred are alkyl (N,N-disubstituted amino)-alkanoates such as C₄ to C₁₋₈ alkyl (N,N-disubstituted amino)-acetates and C₄ to C₁₋₈ alkyl (N,N-disubstituted amino)-propionates and pharmaceutically acceptable salts and derivatives thereof. Exemplary specific alkyl-2-(N,N-disubstituted amino)-alkanoates include dodecyl 2-(N,N dimethylamino)-propionate (DDAIP);

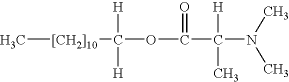

and dodecyl 2-(N,N-dimethylamino)-acetate (DDAA);

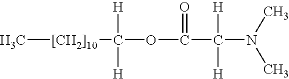

Alkyl-2-(N-substituted amino)-alkanoates are known. For example, dodecyl 2-(N,N-dimethylamino)-propionate (DDAIP) is available from Steroids, Ltd. (Chicago, Ill.). In addition, alkyl-2-(N,N-disubstituted amino)-alkanoates can be synthesized from more readily available compounds as described in U.S. Pat. No. 4,980,378 to Wong et al., which is incorporated herein by reference to the extent that it is not inconsistent. As described therein, alkyl-2-(N,N-disubstituted amino)-alkanoates are readily prepared via a two-step synthesis. In the first step, long chain alkyl chloroacetates are prepared by reaction of the corresponding long chain alkanols with chloromethyl chloroformate or the like in the presence of an appropriate base such as triethylamine, typically in a suitable solvent such as chloroform. The reaction can be depicted as follows:

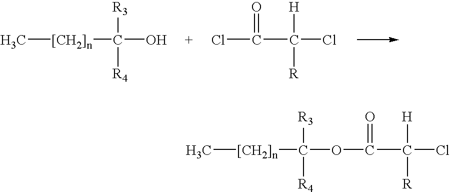

wherein R, R₃, R₄ and n are defined as above. The reaction temperature may be selected from about 10 degrees Celsius to about 200 degrees Celsius or reflux, with room temperature being preferred. The use of a solvent is optional. If a solvent is used, a wide variety of organic solvents may be selected. Choice of a base is likewise not critical. Preferred bases include tertiary amines such as triethylamine, pyridine and the like. Reaction time generally extends from about one hour to three days.

In the second step, the long chain alkyl chloroacetate is condensed with an appropriate amine according to the scheme:

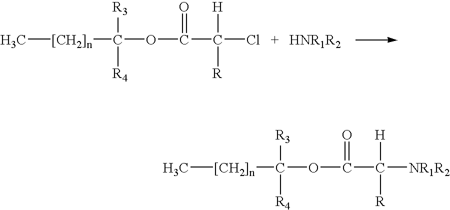

wherein n, R, R₁, R₂, R₃ and R₄ are defined as before. Excess amine reactant is typically used as the base and the reaction is conveniently conducted in a suitable solvent such as ether. This second step is preferably run at room temperature, although temperature may vary. Reaction time usually varies from about one hour to several days. Conventional purification techniques can be applied to ready the resulting ester for use in a pharmaceutical compound.

Suitable (N-substituted amino)-alkanol alkanoates can be represented by the formula:

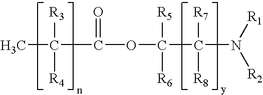

wherein n is an integer having a value in the range of about 5 to about 18; y is an integer having a value in the range of 0 to about 5; and R₁, R₂, R₃, R₄, R₅, R₆, and R₇ are selected from the group consisting of hydrogen, C₁ to C₈ alkyl, and C₃ to C₈ aryl; and R₈ is a selected from the group consisting of hydrogen, hydroxyl, C₁ to C₈ alkyl, and C₃ to C₈ aryl.

Preferred are (N-substituted amino)-alkanol alkanoates such as C₅ to C₁₈ carboxylic acid esters and pharmaceutically acceptable salts thereof. Exemplary specific (N,N-disubstituted amino)-alkanol alkanoates include

1-(N,N-dimethylamino)-2-propanol dodecanoate (DAIPD);

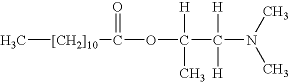

1-(N,N-dimethylamino)-2-propanol myristate (DAIPM);

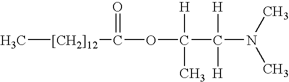

1-(N,N-dimethylamino)-2-propanol oleate (DAIPO);

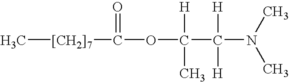

The (N,N-disubstituted amino)-alkanol alkanoates are readily prepared by reacting the corresponding aminoalkinol with lauroyl chloride in the presence of triethylamine. A solvent such as chloroform is optional but preferred. For example, 1-(N,N-dimethylamino)-2-propanol can be reacted with lauroyl chloride in chloroform and in the presence of triethylamine to form 1-(N,N-dimethylamino)-2-propanol dodecanoate (DAIPD).

The penetration enhancer is present in an amount sufficient to enhance the penetration of the prostaglandin E₁. The specific amount varies necessarily according to the desired release rate and the specific form of prostaglandin E₁ used. Generally, this amount ranges from about 0.5 percent to about 10 percent, based on the total weight of the composition. Preferably, the penetration enhancer is about 5 weight percent of the composition.

Additionally, other known transdermal penetration enhancers can also be added, if desired. Illustrative are dimethyl sulfoxide (DMSO), dimethyl acetamide (DMA), 2-pyrrolidone, N,N-diethyl-m-toluamide (DEET), 1-dodecylazacycloheptane-2-one, (Azone™, a registered trademark of Nelson Research), N,N-dimethylformamide, N-methyl-2-pyrrolidone, calcium thioglycolate, oxazolidinone, dioxolane derivatives, laurocapram derivatives, and macrocyclic enhancers such as macrocyclic ketones.

Natural and modified polysaccharide gums are also an important ingredient of the composition. Suitable representative gums are those in the natural and modified galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carobtree (ceratonia siliqua). Suitable modified polysaccharide gums include ethers of natural or substituted polysaccharide gums, such as carboxymethyl ethers, ethylene glycol ethers and propylene glycol ethers. An exemplary substituted polysaccharide gum is methylcellulose.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., N.Y. (1980). Most gums are commercially available in various forms, commonly a powder, and ready for use in foods and topical compositions. For example, locust bean gum in powdered form is available from Tic Gums Inc. (Belcam, Md.).

When present, the polysaccharide gums are present in the range from about 0.1 percent to about 5 percent, based on the total weight of the composition, with the preferred range being from 0.5 percent to 3 percent. In one preferred embodiment, 2.5 percent by weight of a polysaccharide gum is present. Illustrative compositions are given in the examples, below.

An optional alternative to the polysaccharide gum is a polyacrylic acid polymer.

A common variety of polyacrylic acid polymer is known generically as “carbomer.” Carbomer is polyacrylic acid polymers lightly cross-linked with polyalkenyl polyether.

It is commercially available from the B. F. Goodrich Company (Akron, Ohio) under the designation “CARBOPOL™.” A particularly preferred variety of carbomer is that designated as “CARBOPOL 940.” Other polyacrylic acid polymers suitable for use are those commercially available under the designations “Pemulen™” (B. F. Goodrich Company) and “POLYCARBOPHIL™” (A. H. Robbins, Richmond, Va.). The Pemulen™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ enhancer is a polyacrylic acid cross-linked with divinyl glycol.

Where polyacrylic acid polymers are present, they represent about 0.5 percent to about 5 percent of the composition, based on its total weight.

Another important component is a lipophilic component. As used herein “lipophilic component” refers to an agent that is both lipophilic and hydrophilic. One of ordinary skill in the pharmaceutical arts will understand that the lipophilic nature, or “lipophilicity” of a given compound is routinely quantified for comparison to other compounds by using the partition coefficient. The partition coefficient is defined by the International Union of Pure and Applied Chemistry (IUPAC) as the ratio of the distribution of a substance between two phases when the heterogeneous system (of two phases) is in equilibrium; the ratio of concentrations (or, strictly speaking, activities) of the same molecular species in the two phases is constant at constant temperature.

The C₁ to C₈ aliphatic alcohols, the C₂ to C₃₀ aliphatic esters, and their mixtures can serve as lipophilic component. Illustrative suitable alcohols are ethanol, n-propanol and isopropanol, while suitable esters are ethyl acetate, butyl acetate, ethyl laurate, methyl propionate, isopropyl myristate and isopropyl palmitate. As used herein, the term “aliphatic alcohol” includes polyols such as glycerol, propylene glycol and polyethylene glycols. In one embodiment, a mixture of alcohol and ester is preferred, and in particular, a mixture of ethanol and ethyl laurate is preferred.

In one embodiment, the C₂ to C₃₀ aliphatic esters, and their mixtures comprising the lipophilic component include C₈ to C₃₀ aliphatic esters of glycerol selected from the group consisting monoglycerides, diglycerides, triglycerides, and mixtures thereof. Suitable aliphatic esters include glyceryl esters of saturated fatty acids, unsaturated fatty acids and mixtures thereof. Suitable saturated fatty acids include caproic acid, caprylic acid, capric acid, lauric acid, myristic acid, palmitic acid, stearic acid, arachidic acid, behenic acid and lignoceric acid. Suitable unsaturated fatty acids include oleic acid, linoleic acid and linolenic acid. Suitable glyceryl esters include glyceryl monooleate, triolein, trimyristin and tristearin, perferably trimyristin.

The concentration of lipophilic component required necessarily varies according to other factors such as the desired semi-solid consistency and the desired skin penetration promoting effects. Suitably the concentration of lipophilic component is in the range of 0.5 percent to 40 percent by weight based on the total weight of the composition. The preferred topical composition contains lipophilic component in the range of 7 percent to 40 percent by weight based on the total weight of the composition.

Where a mixture of aliphatic alcohol and aliphatic ester are employed, the suitable amount of alcohol is in the range of 0.5 percent to 10 percent. In one preferred embodiment, the amount of alcohol is in the range of 5 percent to 15 percent, while that of aliphatic ester is in the range from 2 percent to 15 percent (again based on the total weight of the composition). In another preferred embodiment, the amount of alcohol is in the range of 0.5 percent to 10 percent, while that of aliphatic ester is in the range from 0 percent to 10 percent (again based on the total weight of the composition).

The concentration of lipophilic component required necessarily varies according to other factors such as the desired semi-solid consistency and the desired skin penetration promoting effects. The preferred topical composition contains lipophilic component in the range of 7 percent to 40 percent by weight based on the total weight of the composition. Where a lipophilic component that is a mixture of aliphatic alcohol and aliphatic ester is used, the preferred amount of alcohol is in the range of 5 percent to 15 percent, while that of aliphatic ester is in the range from 2 percent to 15 percent (again based on the total weight of the composition).

An optional, but preferred, component is an emulsifier. Although not a critical factor, a suitable emulsifier generally will exhibit a hydrophilic-lipophilic balance number greater than 10. Sucrose esters, and specifically sucrose stearate, can serve as emulsifiers for the composition. Sucrose stearate is a well-known emulsifier available from various commercial sources. When an emulsifier is used, sucrose stearate present up to about 2 percent, based on the total weight of the composition, is preferred. The preferred amount of sucrose stearate emulsifier can also be expressed as a weight ratio of emulsifier to polysaccharide gum. A ratio of 1 to 6 emulsifier to gum is preferred, and a ratio of 1 to 4 is most preferred to generate the desired semi-solid consistency and separation resistance.

Other emulsifiers are also suitable including polyoxyethylene sorbitan esters, long chain alcohols, preferably cetostearyl alcohol, and fatty acid glycerides. Suitable polyoxyethylene sorbitan esters include the monolaurate (Tween 20, Span 20) the monopalmitate (Tween 40), the monostearate (Tween 60), and the monooleate (Tween 80) and mixtures thereof. Preferred fatty acid glycerides include glyceryl monooleate, triolein, trimyristin and tristearin.

The composition includes an acid buffer system. Acid buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are countless suitable buffers, potassium phosphate monohydrate has proven effective for compositions of the present invention.

The final pH value of the pharmaceutical composition may vary within the physiologically compatible range. Necessarily, the final pH value is not irritating to human skin. Without violating this constraint, the pH may be selected to improve prostaglandin E₁ stability and to adjust consistency when required. In one embodiment, the preferred pH value is about 3.0 to about 7.4, more preferably about 3.0 to about 6.5, most preferably from about 3.5 to about 6.0.

The remaining component of the composition is water, which is necessarily purified. The composition contains water in the range of about 50 to about 90 percent, based on the total weight of the composition. The specific amount of water present is not critical, however, being adjustable to obtain the desired consistency and/or concentration of the other components.

Prostaglandin E₁ stabilizers, coloring agents, rheological agents, and preservatives can be added to the extent that they do not overly limit prostaglandin E₁ skin penetration or prevent the desired semi-solid consistency.

Contemplated dosage forms of the semi-solid pharmaceutical composition are creams, gels, ointments, colloidal suspensions and the like, also including but not limited to compositions suitable for use with transdermal patches and like devices.

The ingredients listed above may be combined in any order and manner that produces a stable composition comprising a prostaglandin E₁ evenly dispersed throughout a semi-solid formulation. One available approach to preparing such compositions involves evenly dispersing the polysaccharide gum (or polyacrylic acid polymer) in a premixed water/buffer solution and then thoroughly homogenizing (i.e. mixing) the resulting mixture, which will be labelled “Part A.” When present, the emulsifier is added to the water/buffer solution before dispersing the polysaccharide gum. Any suitable method of adjusting the pH value of Part A to the desired level may be used, for example, by adding concentrated phosphoric acid or sodium hydroxide.

Separately, the prostaglandin E₁ is dissolved with agitation in the lipophilic component, which itself may be a mixture of alcohols, esters, or alcohol with ester. Next, the penetration enhancer is added. Alternatively, when the lipophilic component includes both an alcohol and an ester, the prostaglandin E₁ can be dissolved in the alcohol before adding the penetration enhancer followed by the ester. In either case, the resulting mixture will be labelled “Part B.” The final step involves slow addition (e.g. dropwise) of Part B into Part A under constant mixing.

The resulting topical composition, when compared to exhibits the advantageous properties described above, including improved prostaglandin E₁ permeation and bioavailability without drug overloading, reduced skin damage and related inflammation, and increased flexibility in design of dosage forms. These compositions can be used for prolonged treatment of peripheral vascular disease, male impotency and other disorders treated by prostaglandin E₁, while avoiding the low bioavailability and rapid chemical decomposition associated with other delivery methods. Application of prostaglandin E₁ in a topical composition to the skin of a patient allows a predetermined amount of prostaglandin E₁ to be administered continuously to the patient and avoids undesirable effects present with a single or multiple administrations of larger dosages by injection. By maintaining a sustained dosage rate, the prostaglandin E₁ level in the patient's target tissue can be better maintained within the optimal therapeutic range.

In one embodiment, a composition comprises about 0.01 percent to about 5 percent modified polysaccharide gum; about 0.001 percent to about 1 percent of a prostaglandin selected from the group consisting of PGE₁, pharmaceutically acceptable salts thereof, lower alkyl esters thereof and mixtures thereof; about 0.5 percent to about 10 percent DDAIP or salts thereof; about 0.5 percent to about 10 percent of a lower alcohol selected from the group consisting of ethanol, propanol, isopropanol and mixtures thereof; about 0.5 percent to about 10 percent on an ester selected from the group consisting of ethyl laurate, isopropyl myristate, isopropyl laurate and mixtures thereof; based on the weight of the composition, and an acid buffer. Preferably the composition also comprises up to about 2 percent sucrose stearate.

Optionally the composition also comprises up to about 5 percent emulsifier. Preferably, the composition also comprises up to about 2 percent emulsifier. Suitable emulsifiers include polysorbates such as Tweens, glyceryl monooleate, triolein, trimyristin and tristearin. A preferred emulsifier is trimyristin.

The practice of the present invention is demonstrated in the following examples. These examples are meant to illustrate the invention rather than to limit its scope. Variations in the treating compositions which do not adversely affect the effectiveness of prostaglandin E₁ will be evident to one skilled in the art, and are within the scope of this invention. For example, additional ingredients such as coloring agents, anti-microbial preservatives, emulsifiers, perfumes, prostaglandin stabilizers, and the like may be included in the compositions as long as the resulting composition retains desirable properties, as described above. When present, preservatives are usually added in amounts of about 0.05 to about 0.30%. Suitable preservatives include methylparabens (methyl PABA), propylparabens (propyl PABA) and butylhydroxy toluene (BHT). Suitable perfumes and fragrances are known in the art; a suitable fragrance is up to about 5 percent myrtenol, preferably about 2 percent myrtenol, based on the total weight of the composition. The compositions of the present invention can also include a small amount, about 0.01 to about 4% by weight, of a topical anesthetic, if desired. Typical topical anesthetics include lidocaine, dyclonine, dibucaine, pharmaceutically acceptable salts and mixtures thereof. In one preferred embodiment, the topical anesthetic is about 0.5 percent dyclonine, based on the weight of the composition.

The pharmaceutical preparation is preferably in unit dosage form. In such form the preparation is subdivided into unit doses containing appropriate quantities of the active component. The unit dosage form is a packaged preparation, where the package containing the discrete quantities of the pharmaceutical preparation is, e.g. a rigid plastic dispenser or flexible packet.

Another aspect of the invention is an article of manufacture that comprises a composition for treating erectile dysfunction as described above in a suitable container, preferably in a container such as the dispenser disclosed in U.S. Pat. No. 6,224,573, in combination with labeling instructions. Alternatively, the container can be a tube with a suitable orifice size, such as an extended tip tube, pouch, packet, or squeeze bottle and made of any suitable material, for example rigid plastic or flexible plastic.

The labeling instructions can come in the form of a pamphlet, a label applied to or associated with the packaging of the article of manufacture.

The labeling instructions provide for administering a composition of the invention to the fossa navicularis of the penis of a patient suffering from erectile dysfunction, directing the patient to hold the penis upright, hold the meatus open and place the composition in the fossa navicularis without introducing the container into the meatus about 5-30 minutes, before sexual intercourse. Printed labeling instructions are functionally related to the composition of the invention inasmuch as such labeling instructions describe a method to treat erectile dysfunction according to the present invention. The labeling instructions are an important aspect of the invention in that before a composition can be approved for any particular use, it must be approved for marketing by the responsible national regulatory agency, such as the United States Food and Drug Administration. Part of that process includes providing a label that will accompany the pharmaceutical composition which is ultimately sold. While the label will include a definition of the composition and such other items such as the clinical pharmacology, mechanism of action, drug resistance, pharmacokinetics, absorption, bioavailability, contraindications and the like, it will also provide the necessary dosage, administration and usage. Thus, the combination of the composition with the dispenser with appropriate treatment instructions is important for the proper usage of the drug once it is marketed to the patient. Such treatment instructions will describe the usage in accordance with the method of treatment set forth herein before.

The quantity of active component in a unit dose preparation may be varied or adjusted from 0.01 mg to 1 g according to the particular application and the potency of the vasoactive prostagladin. For example, where the vasoactive prostaglandin is prostaglandin E₁, about 0.05 mg to about 0.8 mg prostaglandin E₁ is present, preferably about 0.1 mg to about 0.5 mg and in another embodiment, about 0.2 mg to about 0.3 mg. The composition can, if desired, also contain other compatible therapeutic agents, such as a piperazinyl quinazoline antihypertensive.

The semi-solid vasoactive prostaglandin composition should be applied to the fossa navicularis of the penis about 2-30 minutes before sexual intercourse, preferably about 10-20 minutes before sexual intercourse.

Unless otherwise indicated, each composition is prepared by conventionally admixing the respective indicated components together.

### EXAMPLE 1

Exemplary Compositions

Exemplary Composition A was prepared as follows. Part A was formed by dissolving 0.4 parts prostaglandin E₁ (Alprostadil USP) in 5 parts ethyl alcohol. Next, 5 parts dodecyl 2-(N,N-dimethylamino)-propionate were mixed into the alcohol-prostaglandin E₁ solution, followed by 5 parts ethyl laurate.

Part B was prepared starting from a pH 5.5 water/buffer solution. The water/buffer solution was prepared by adding sufficient potassium phosphate monohydride to purified water to create a 0.1 M solution. The pH of the water/buffer solution was adjusted to 5.5 with a strong base solution (1 N sodium hydroxide) and a strong acid (1 N phosphoric acid). The buffer solution represented about 80 parts of the total composition. All parts specified herein are parts by weight.

To the buffer solution, was added 0.5 parts ethyl laurate. Next, the locust bean gum (in powder form) was dispersed in the buffer solution and homogenized using a homogenizer. Table 1, below, contains a list of ingredients.

The resulting composition was a spreadable, semi-solid suitable for application to the skin without the need for supporting devices such as patches and adhesive strips. The composition was both homogenous in appearance and resistant to separation.

TABLE 1
Topical Prostaglandin E₁ Compositions

| Ingredient | | | | | | | | |
|---|---|---|---|---|---|---|---|---|
| (wt %) | A | B | C | D | E | F | G | H |
| Part A: | | | | | | | | |
| prehydrated | 3 | 3 | 3 | 3 | 3 | 3 | 3 | — |
| locust | | | | | | | | |
| bean gum | | | | | | | | |
| prehydrated | — | — | — | — | — | — | — | 3 |
| modified | | | | | | | | |
| guar gum | | | | | | | | |
| water/buffer | 81 | 81 | 81 | 81 | 81 | 81 | 81 | 81 |
| (pH 5.5) | | | | | | | | |
| sucrose stearate | 0.5 | 0.5 | 0.5 | 0.5 | 0.5 | 0.5 | 0.5 | — |
| Part B: | | | | | | | | |
| prostaglandin | 0.1 | 0.2 | 0.3 | 0.4 | 0.4 | 0.5 | 0.4 | 0.3 |
| E₁ | | | | | | | | |
| DDAIP | 5 | 5 | 5 | 5 | 5 | 5 | 5 | 2.5 |
| ethanol | 5 | 5 | 5 | 5 | 5 | 5 | 10 | 5 |
| ethyl laurate | 5 | 5 | 5 | 5 | 5 | 5 | — | 3 |

Additional exemplary compositions B-H were prepared in the same manner using the components listed in Table 1. As noted above, in other embodiments, such as Composition H, the composition may include a modified polysaccharide gum, suitably a modified galactomannan gum, such as a modified guar gum. Alternatively, a polyacrylic acid polymer may be used instead of the polysaccharide gum.

### EXAMPLE 2

Administration of an Exemplary Composition to Healthy Volunteers

A semi-solid prostaglandin composition, Composition H, was used to treat healthy volunteers using the method of the present invention. It was found that placement of a composition comprising an amount of a semi-solid prostaglandin composition in the fossa navicularis results in an increase in blood flow in the glans and an increased intumescence of the penis. In preferred embodiments, the treatment of healthy subjects results in intumescence of the glans, and preferably a penile erection. The study was performed on a population of eight healthy volunteers without erectile dysfunction.

Some relevant characteristics of the study group are summarized in Table 2. Eight healthy volunteers not suffering from ED ranged in age from 27 to 64 years old (41.1±11.9, mean±standard deviation). Additional information regarding the result of treatment is provided for each healthy volunteer in Table 2.

Evaluation was performed based on the individual's subjective evaluation. Briefly, when the administration of the composition achieved an erection sufficient to facilitate sexual intercourse based on the individual's sexual experience, patients reported the result as (+). When erection was insufficient or only intumescence was obtained, patients reported the result as (+). When there were no changes, patients reported the result as (−). The results are reported in this manner in Table 2.

The study participants were instructed to place the medication in the fossa navicularis by holding the penis upright, holding the meatus open and dropping the medication into the fossa navicularis without introducing the medication container into the meatus.

All eight subjects in the healthy volunteer group showed increased intumescence (increases in width and length of their penises) after a single dose of Composition H of Example 1 without erotic stimulation. One participant experienced a full erection and seven participants experienced intumescence that lasted from 60 to 180 minutes in duration. See Table 2, below. Adverse events were all localized to the site of application and short in duration.

TABLE 2
Individual Findings: Healthy Volunteers

| | | | | | Duration of | |
|---|---|---|---|---|---|---|
| | | | Side Effect | Systemic | Erection or | |
| Volunteer | Age | Result | (Local pain) | Side Effect | Intumescence | Note |
| 1 | 27 | ± | Mild, | None | About 2.5 hours | Ejaculation occurred |
| | | | Transient | | | |
| 2 | 33 | + | Mild, | None | About 3 hours | Ejaculation occurred; |
| | | | Transient | | | erection maintained |
| | | | | | | after ejaculation |
| 3 | 35 | ± | Mild, | None | About 2 hours | Ejaculation occurred |
| | | | Transient | | | |
| 4 | 35 | ± | None | None | About 2 hours | Ejaculation occurred |
| 5 | 38 | ± | Mild, | None | 1.75 hours | Ejaculation occurred |
| | | | Transient | | | |
| 6 | 46 | ± | Mild, | None | 1.25 hours | NA |
| | | | Transient | | | |
| 7 | 51 | ± | None | None | 1 hour | NA |
| 8 | 64 | ± | Mild, | None | 1.5 hours | NA |
| | | | Transient | | | |

Summary: Each participant received one to four doses; all participants reported an effect of treatment: seven (±), one (+); duration one hour or more in all patients. NA = not applicable.

### EXAMPLE 3

Administration of an Exemplary Composition to ED Patients

A semi-solid prostaglandin composition, Composition H, was used to treat ED in a group of patients needing such treatment using the method of the present invention. It was found that placement of a composition comprising an amount of a semi-solid prostaglandin composition in the fossa navicularis results in an increase in blood flow in the glans and an increased intumescence of the penis. In preferred embodiments, the treatment results in intumescence of the glans, and preferably a penile erection. The study was performed on a population of thirteen ED patients.

Relevant characteristics of the study group are summarized in Table 3. Thirteen ED patients ranging from 33 to 68 years old (51.0±10.3, mean±standard deviation) were screened, and achieved a full erection with an oral dose of 50 mg sildenafil citrate (Viagra™). The severity of erectile dysfunction was assessed for each patient using the 5-Item International Index of Erectile Function (IIEF-5), a standard five item questionnaire in which lower scores on a scale of 0-25 indicate a greater degree of erectile dysfunction (Rosen R C, Cappelleri J C, Smith M D, Lipsky J, Peña B M. Development and evaluation of an abridged, 5-item version of the International Index of Erectile Function (IIEF-5) as a diagnostic tool for erectile dysfunction. Int. J. Impot. Res. December 1999; 11(6):319-26). Additional information regarding the result of treatment is provided for each ED patient in Table 3.

The patients were instructed to place the medication in the fossa navicularis by holding the penis upright, holding the meatus open and dropping the medication into the fossa navicularis without introducing the medication container into the meatus.

Evaluation of treatment results was performed based on the individual's subjective evaluation. Briefly, when the administration of the composition achieved an erection sufficient to facilitate sexual intercourse based on the individual's sexual experience, patients reported the result as (+). When erection was insufficient or only intumescence was obtained, patients reported the result as (±). When there were no changes, patients reported the result as (−). The results are reported in this way in Table 3.

In the ED group, a full erection was attained by seven patients after treatment followed by erotic stimulation (audio-visual sexual stimulation (“AVSS”), i.e., viewing erotic videotapes). Another four patients experienced some degree of tumescence after treatment followed by erotic stimulation, while the remaining two patients did not have a noticeable change. See Table 3, below.

Adverse events were all localized to the site of application and short in duration; no systemic side effects were observed. Two patients withdrew from the study due to local irritation that did not need medical attention. An erection sufficient for intercourse was achieved by the majority of the ED patients (7/13) after treatment with one dose of the topical composition H that contains 0.3 mg prostaglandin and followed by erotic stimulation. An additional four patients had some tumescence in response to the same treatment, and only two of thirteen patients experienced no change.

TABLE 3
Individual Findings: ED Patients

| | | IIEF-5 | Re- | Side Effect | |
|---|---|---|---|---|---|
| Pt. | Age | Score | sult | (Local pain) | Underlying Disease |
| 1 | 33 | 5 | ± | + | Type I Diabetes mellitis |
| | | | | Edema | |
| 2 | 42 | 5 | ± | + | Depression |
| 3 | 43 | 7 | + | ± | Organic ED & ejaculation |
| | | | | | disturbance after surgery for |
| | | | | | rectal cancer |
| 4 | 44 | 5 | + | + | Psychogenic ED |
| 5 | 47 | 10 | + | ± | PsychogenicED, male |
| | | | | | infertility |
| 6 | 49 | 9 | ± | + | Organic ED |
| 7 | 50 | 6 | ± | − | Type II Diabetes mellitis (8 |
| | | | | | year history) |
| 8 | 51 | 9 | − | − | Psychogenic ED |
| 9 | 52 | 7 | + | − | Psychogenic ED |
| 10 | 52 | 12 | + | ± | Diabetes mellitis, hepatitis C |
| 11 | 65 | 11 | + | − | Prostatic hypertrophy |
| 12 | 67 | 15 | + | ± | Psychogenic ED |
| 13 | 68 | 15 | − | ++ | Prostatic hypertrophy |

Summary: Each patient received one to four doses; seven patients (+), four patients (±), two patients (−). Side effects such as local pain, nine patients; systemic side effects, None.

### EXAMPLE 4

Objective Measurements of Increases in Blood Flow

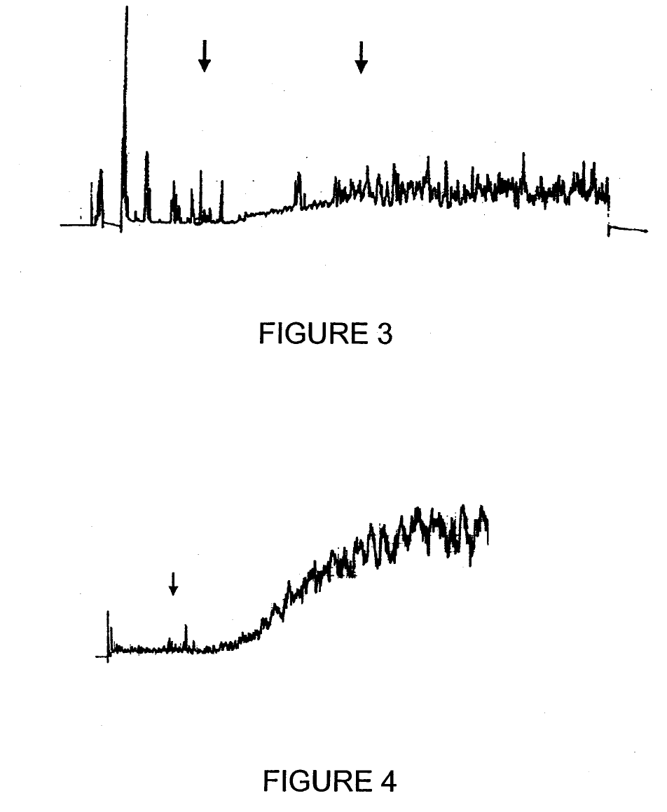

Objective measures of the increases in glans microcirculation were obtained using a laser doppler flowmeter (PeriFlux System 5000, Perimed, Sweden). An example of the recordings obtained is shown in FIG. 3. The degree of ED in each patient was rated using the five item form of the International Index of Erectile Dysfunction (“IIEF-5”). A summary of the patients and the results is presented in Table 4, below.

FIG. 3 is a graphical representation of the results of a laser doppler flowmeter measurement of the blood flow in the glans penis of a 65 year old patient not exhibiting erectile dysfunction (IIEF-5 score: 24, BP 137/82) before, during and after treatment using the method of the present invention with a dose of Composition H comprising 0.3 mg prostaglandin E₁ at the time indicated by the arrow, showing an increase in blood flow following the treatment (left arrow) followed by erection at 14 minutes (right arrow).

FIG. 4 is a graphical representation of the results of a laser doppler flowmeter measurement of the blood flow in the glans penis of a 51 year old erectile dysfunction patient (IIEF-5 score: 10, severe ED) before, during and after treatment using the method of the present invention with a dose of Composition H comprising 0.2 mg prostaglandin E₁ at the time indicated by the arrow, showing an increase in blood flow following the treatment, with the glans reaching tumescence in 9 minutes.

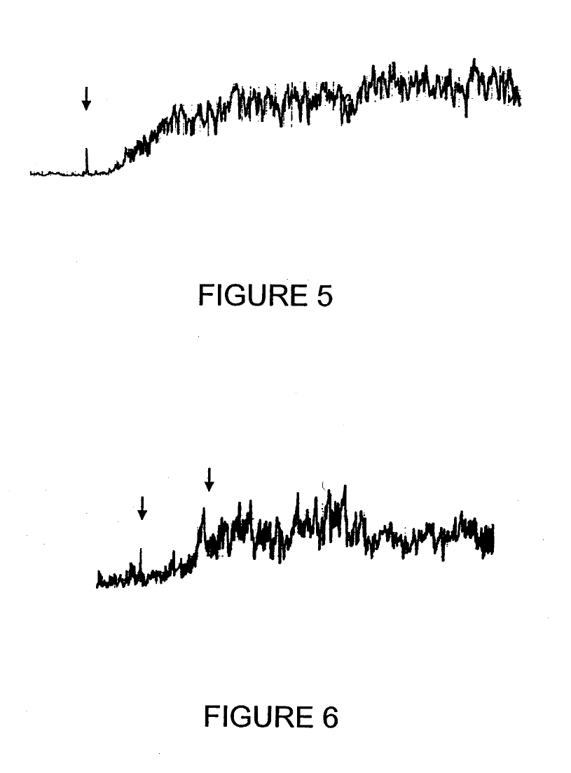

FIG. 5 is a graphical representation of the results of a laser doppler flowmeter measurement of the blood flow in the glans penis of a 57 year old erectile dysfunction patient (IIEF-5 score: 15, moderate ED, BP 124/50) before, during and after treatment using the method of the present invention with a dose of Composition H comprising 0.2 mg prostaglandin E₁ at the time indicated by the arrow, showing an increase in blood flow following the treatment, with the glans reaching tumescence in 4 minutes.

FIG. 6 is a graphical representation of the results of a laser doppler flowmeter measurement of the blood flow in the glans penis of a 56 year old erectile dysfunction patient (IIEF-5 score: 9, severe ED, BP 144/88) before, during and after treatment using the method of the present invention with a dose of Composition H comprising 0.2 mg prostaglandin at the time indicated by the left arrow, showing an increase in blood flow following the treatment, with the glans reaching tumescence in 7 minutes (right arrow).

TABLE 4
Measurement of Glans Microcirculation by Laser Dopper Flowmeter

| | | | Time to Glans | Rigid | | |
|---|---|---|---|---|---|---|
| | | IIEF-5 | Tumescence | Erection | | |
| Pt. | Age | Score | (Minutes) | With AVSS | Underlying Disease | PGE1 Dose (mg) |
| 14 | 65 | 24 | 14 | + | Age-related | 0.3 |
| 15 | 57 | 15 | 5 | + | Psychogenic ED | 0.2 |
| 16 | 56 | 9 | About 7 | ± | Mixed ED | 0.2 |
| 17 | 51 | 10 | About 8 | + | Psychogenic ED | 0.2 |
| 18 | 62 | 2 | 9 | − | Depression | 0.2 |
| 19 | 45 | 13 | 2 | + | Psychogenic ED | 0.2 |
| 20 | 29 | 7 | 4.5 | − | Severe Neurosis | 0.2 |
| 21 | 61 | 4 | About 7 | − | Mixed ED | 0.2 |
| 22 | 62 | 4 | 4 | − | Psychogenic ED | 0.2 |
| 23 | 60 | 14 | 6 | + | Psychogenic ED | 0.2 |

Summary: Rigid erection with audio-visual sexual stimuli (AVSS) 5 patients (+), (±) 1 patient, (−) 4 patients; duration to glans tumescence 2-14 minutes; no adverse events.

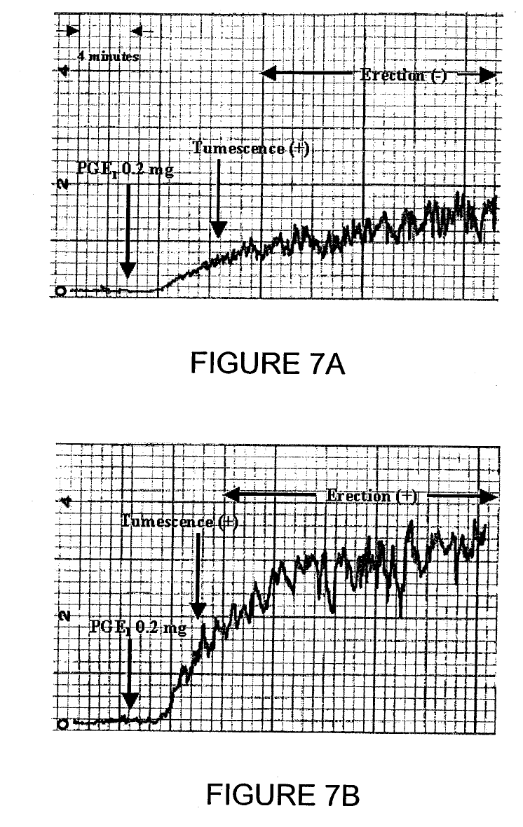

FIGS. 7A and 7B are graphical representations of the results of a laser doppler flowmeter measurement of the blood flow in the glans penis of a 60 year old erectile dysfunction patient (IIEF-5 score: 14, moderate ED, BP 145/88) before, during and after treatment using the method of the present invention with a semisolid composition comprising 0.2 mg prostaglandin E₁ FIG. 7A: 0.2 mg prostaglandin E₁ was administered in the absence of sexual stimuli (“SS(−)”) at the time indicated by the arrow, showing an increase in blood flow following the treatment, with the glans reaching tumescence in about 6 minutes (arrow). Glans bloodflow increased to the level expected during a physiologically normal erection (roughly six fold of baseline) within ten minutes, but no erection developed under the conditions and surroundings of the study in the absence of sexual stimulation.

In FIG. 7B, the same dose of prostaglandin E₁ administered in the presence of audio-visual erotic stimuli (“AVSS(+)”) produced a larger and more rapid increase in glans blood flow and tumescence. A rigid erection was obtained that was maintained for more than one hour.

Without being held to a particular mechanism, it is believed that the treatment of the present invention comprising placing a semisolid prostaglandin composition into the fossa navicularis results in the permeation of prostaglandin into the tissue of the glans penis and into the corpus spongiosum. The effect of prostaglandin E₁ in the glans produces a prompt increase in blood flow followed by tumescence of the glans and the penis as a whole. In the presence of audio-visual erotic stimulation, the penile tumescence of ED patients can progress to a maintained erection adequate for intercourse.

In the study of Example 4, administration of a composition comprising 0.3 mg prostaglandin E₁ in the presence of audio-visual erotic stimulation resulted in an erection sufficient for intercourse in the majority of the ED patients (7/13); an additional four ED patients had some tumescence in response to the same treatment. Only two of the thirteen ED patients experienced no change. Administration of the 0.3 mg prostaglandin E₁ dose to a patient not suffering from ED (IIEF-5 score: 24, patient 14, Table 4 and FIG. 3) in the presence of audio-visual erotic stimuli resulted in an erection suitable for intercourse.

The protocol in the study of Example 5 was modified to add objective measurements of glans microcirculation. A lower dose of prostaglandin E₁ (0.2 mg), was administered to the ED patients. In the presence of audio-visual sexual stimulation, the administration of this lower dose of prostaglandin E₁ resulted in penile tumescence that progressed to an erection sufficient for intercourse in 4 of 9 ED patients. Another ED patient had some tumescence in response to the same treatment, and only two of nine ED patients experienced no change. Thus, administration of the lower dose of prostaglandin E₁ resulted in a lower proportion of the treated ED patients attaining an erection sufficient for intercourse, while a larger proportion of the ED patients showed tumescence not sufficient for intercourse or no change.

Without being held to a particular mechanism, it is believed that the erotic stimulation may act though a nitric oxide (“NO”) mediated system in combination with treatment with the prostaglandin E₁ composition of the present invention to produce an erection in the ED patient.

We claim:

1. A method of treating erectile dysfunction in a patient needing such treatment comprising the steps:
placing in the fossa navicularis of the patient an amount of a semi-solid vasoactive prostaglandin composition effective to increase blood flow in the glans penis; comprising
a vasoactive prostaglandin;
a penetration enhancer;
a polymer selected from the group consisting of polysaccharide gums and polyacrylic acid polymers;
a lipophilic component that is selected from the group consisting of an aliphatic C₁ to C₈ alcohol, an aliphatic C₈ to C₃₀ ester, and a mixture thereof, an acidic buffer system; and
providing at least one erotic stimulus selected from the group consisting of olfactory stimuli, visual stimuli, auditory stimuli and tactile stimuli.

2. The method in accordance with claim 1 wherein the vasoactive prostaglandin is selected from the group consisting of PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF₃ and mixtures thereof.

3. The method in accordance with claim 1 wherein the vasoactive prostaglandin is prostaglandin E₁.

4. The method of claim 1 wherein the vasoactive prostaglandin is present in the amount of about 0.1 mg to about 0.5 mg.

5. The method of claim 1 wherein the vasoactive prostaglandin is present in the amount of about 0.2 mg to about 0.3 mg.

6. The method in accordance with claim 1 wherein the polymer is a polyacrylic acid polymer.

7. The method in accordance with claim 1 wherein the polymer is a shear-thinning polysaccharide gum.

8. The method in accordance with claim 7 wherein the shear-thinning polysaccharide gum is a galactomannan gum.

9. The method in accordance with claim 7, wherein the shear-thinning polysaccharide gum is a modified galactomannan gum.

10. The method in accordance with claim 9 wherein the modified galactomannan gum is a modified guar gum.

11. The method in accordance with claim 1 wherein the penetration enhancer is selected from the group consisting of an alkyl-2-(N-substituted amino)-alkanoate, an (N-substituted)-alkanol alkanoate, pharmaceutically acceptable salts thereof and mixtures thereof.

12. The method in accordance with claim 11 wherein the penetration enhancer is dodecyl 2-(N,N-dimethylamino)-propionate.

13. The method in accordance with claim 1 wherein the lipophilic component comprises at least one aliphatic C₈ to C₃₀ ester.

14. The method in accordance with claim 1 wherein the lipophilic component comprises at least one glyceryl ester selected from the group consisting monoglycerides, diglycerides, triglycerides, and mixtures thereof.

15. The method in accordance with claim 1 wherein the lipophilic component comprises at least one glyceryl ester selected from the group consisting of glyceryl monooleate, triolein, trimyristin, tristearin, and mixtures thereof.

16. The method in accordance with claim 1 wherein the acidic buffer system provides a buffered pH value for said composition in the range of about 3 to about 6.5.

17. The method in accordance with claim 1 wherein the composition further comprises an emulsifier selected from the group consisting of sucrose esters, polyoxyethylene sorbitan esters, long chain alcohols, and glyceryl esters.

18. The method in accordance with claim 1 wherein the emulsifier comprises at least one glyceryl ester selected from the group consisting of glyceryl monooleate, triolein, trimyristin, tristearin, and mixtures thereof.

19. The method in accordance with claim 1 wherein the composition further comprises up to about 5 percent myrtenol, based on the total weight of the composition.

20. The method in accordance with claim 1 wherein the composition further comprises a preservative.

21. The method in accordance with claim 1 wherein the composition further comprises a topical anesthetic.

22. The method in accordance with claim 1 wherein the composition further comprises a fragrance.

23. A method of improving microcirculation in the glans penis in a patient needing such treatment comprising:
placing in the fossa navicularis of the patient an amount of a semi-solid vasoactive prostaglandin composition effective to increase blood flow in the glans penis; comprising
a vasoactive prostaglandin;
a penetration enhancer that is chosen from the group consisting of an alkyl-2-(N-substituted amino)-alkanoate, an (N-substituted)-alkanol alkanoate, pharmaceutically acceptable salts thereof and a mixture thereof;
a polysaccharide gum;
a lipophilic component that is a selected from the group consisting of an aliphatic C₁ to C₈ alcohol, an aliphatic C₈ to C₃₀ ester, and a mixture thereof; and
an acidic buffer system.

24. The method in accordance with claim 23 wherein the vasoactive prostaglandin is selected from the group consisting of PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF₃ and mixtures thereof.

25. The method in accordance with claim 23 wherein the vasoactive prostaglandin is prostaglandin E₁.

26. The method in accordance with claim 23 wherein the polysaccharide gum is a shear-thinning polysaccharide gum.

27. The method in accordance with claim 23 wherein the shear-thinning polysaccharide gum is a galactomannan gum.

28. The method in accordance with claim 26 wherein the shear-thinning polysaccharide gum is a modified galactomannan gum.

29. The method in accordance with claim 28 wherein the modified galactomannan gum is a modified guar gum.

30. The method in accordance with claim 23 wherein the penetration enhancer is dodecyl 2-(N,N-dimethylamino)-propionate.

31. The method in accordance with claim 23 wherein the lipophilic component comprises at least one aliphatic C₈ to C₃₀ ester.

32. The method in accordance with claim 23 wherein the lipophilic component comprises at least one glyceryl ester selected from the group consisting monoglycerides, diglycerides, triglycerides, and mixtures thereof.

33. The method in accordance with claim 23 wherein the lipophilic component comprises at least one glyceryl ester selected from the group consisting of glyceryl monooleate, triolein, trimyristin, tristearin, and mixtures thereof.

34. The method in accordance with claim 23 wherein the acidic buffer system provides a buffered pH value for said composition in the range of about 3 to about 6.5.

35. The method in accordance with claim 23 wherein the composition further comprises an emulsifier selected from the group consisting of sucrose esters, polyoxyethylene sorbitan esters, long chain alcohols, and glyceryl esters.

36. The method in accordance with claim 23 wherein the emulsifier comprises at least one glyceryl ester selected from the group consisting of glyceryl monooleate, triolein, trimyristin, tristearin, and mixtures thereof.

37. The method in accordance with claim 23 wherein the composition further comprises a fragrance.

38. The method in accordance with claim 23 wherein the composition further comprises up to about 5 percent myrtenol, based on the total weight of the composition.

39. The method in accordance with claim 23 wherein the composition further comprises a preservative.

40. The method in accordance with claim 23 wherein the composition further comprises a topical anesthetic.

41. A method of treating erectile dysfunction in a patient needing such treatment comprising the steps:
placing in the fossa navicularis of the patient an amount of a semi-solid vasoactive prostaglandin composition effective to increase blood microcirculation in the glans penis; comprising
a vasoactive prostaglandin;
a penetration enhancer that is selected from the group consisting of an alkyl-2-(N-substituted amino)-alkanoate, an (N-substituted)-alkanol alkanoate, pharmaceutically acceptable salts thereof and a mixture thereof;
a polyacrylic acid polymer;
a lipophilic component which is selected from the group consisting of an aliphatic C₁ to C₈ alcohol, an aliphatic C₈ to C₃₀ ester, and a mixture thereof, an acidic buffer system; and
providing at least one erotic stimulus selected from the group consisting of olfactory stimuli, visual stimuli, auditory stimuli and tactile stimuli.

42. The method in accordance with claim 41 wherein the vasoactive prostaglandin is selected from the group consisting of PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF₃ and mixtures thereof.

43. The method in accordance with claim 41 wherein the vasoactive prostaglandin is prostaglandin E_{1.}

44. The method in accordance with claim 41 wherein the polyacrylic acid polymer is a shear-thinning polyacrylic acid polymer.

45. The method in accordance with claim 41 wherein the penetration enhancer is dodecyl 2-(N,N-dimethylamino)-propionate.

46. The method in accordance with claim 41 wherein the lipophilic component comprises at least one aliphatic C₈ to C₃₀ ester.

47. The method in accordance with claim 41 wherein the lipophilic component comprises at least one glyceryl ester selected from the group consisting monoglycerides, diglycerides, triglycerides, and mixtures thereof.

48. The method in accordance with claim 41 wherein the lipophilic component comprises at least one glyceryl ester selected from the group consisting of glyceryl monooleate, triolein, trimyristin, tristearin, and mixtures thereof.

49. The method in accordance with claim 41 wherein the acidic buffer system provides a buffered pH value for said composition in the range of about 3 to about 6.5.

50. The method in accordance with claim 41 wherein the composition further comprises an emulsifier selected from the group consisting of sucrose esters, polyoxyethylene sorbitan esters, long chain alcohols, and glyceryl esters.

51. The method in accordance with claim 41 wherein the emulsifier comprises at least one glyceryl ester selected from the group consisting of glyceryl monooleate, triolein, trimyristin, tristearin, and mixtures thereof.

52. The method in accordance with claim 41 wherein the composition further comprises up to about 5 percent myrtenol, based on the total weight of the composition.

53. The method in accordance with claim 41 wherein the composition further comprises a preservative.

54. The method in accordance with claim 41 wherein the composition further comprises a topical anesthetic.

55. A method of producing tumescence of the glans penis in a patient needing such treatment comprising the steps of:
placing in the fossa navicularis of the patient an amount of a semi-solid vasoactive prostaglandin composition effective to increase blood microcirculation in the glans penis, the composition comprising
a vasoactive prostaglandin;
a penetration enhancer that is selected from the group consisting of an alkyl-2-(N-substituted amino)-alkanoate, an (N-substituted)-alkanol alkanoate, pharmaceutically acceptable salts thereof and a mixture thereof;
a polymer selected from the group consisting of polysaccharide gums and polyacrylic acid polymers;
a lipophilic component which is selected from the group consisting of an aliphatic C₁ to C₈ alcohol, an aliphatic C₈ to C₃₀ ester, and a mixture thereof, and
an acidic buffer system; thereby producing tumescence of the glans penis.

56. The method of claim 55 further producing tumescence of the corpora cavernosa.

57. A method of treating erectile dysfunction in a patient needing such treatment comprising the steps of:
placing in the fossa navicularis of the patient an amount of a semi-solid vasoactive prostaglandin composition effective to increase blood microcirculation in the glans penis, the composition comprising
about 0.05 mg to about 0.8 mg of prostaglandin E₁;
a penetration enhancer that is selected from the group consisting of an alkyl-2-(N-substituted amino)-alkanoate, an (N-substituted)-alkanol alkanoate, pharmaceutically acceptable salts thereof and a mixture thereof,
a polymer selected from the group consisting of polysaccharide gums and polyacrylic acid polymers;
a lipophilic component that is selected from the group consisting of an aliphatic C₁ to C₈ alcohol, an aliphatic C₈ to C₃₀ ester, and a mixture thereof, an acidic buffer system; and
providing at least one erotic stimulus selected from the group consisting of olfactory stimuli, visual stimuli, auditory stimuli and tactile stimuli.

58. The method of claim 57 wherein prostaglandin is present in the amount of about 0.1 mg to about 0.5 mg.

59. The method of claim 57 wherein prostaglandin E₁ is present in the amount of about 0.2 mg to about 0.3 mg.

60. An article of manufacture comprising an unit dose of a composition comprising a vasoactive prostaglandin, a penetration enhancer, a polymer selected from the group consisting of polysaccharide gums and polyacrylic acid polymers, a lipophilic component and an acidic buffer in a suitable container in combination with labeling instructions.

61. The article of manufacture of claim 60 wherein the vasoactive prostaglandin is prostaglandin E₁ present in the amount of about 0.05 mg to about 0.8 mg.

62. The article of manufacture of claim 60 wherein the vasoactive prostaglandin is prostaglandin E₁ present in the amount of about 0.1 mg to about 0.5 mg.

63. The article of manufacture of claim 60 wherein the vasoactive prostaglandin is prostaglandin E₁ present in the amount of about 0.2 mg to about 0.3 mg.

64. A composition comprising:
an amount of a vasoactive prostaglandin effective to increase blood flow in the glans penis;
a piperazinyl quinazoline antihypertensive;
a penetration enhancer;
a polymer selected from the group consisting of polysaccharide gums and polyacrylic acid polymers;
a lipophilic component selected from the group consisting of an aliphatic C₁ to C₈ alcohol, an aliphatic C₈ to C₃₀ ester, and a mixture thereof; and
an acidic buffer system.

65. The composition of claim 64 wherein the piperazinyl quinazoline antihypertensive is selected from the group consisting of alfuzosin, bunazosin, doxazosin, prazosin, terazosin, trimazosin and mixtures thereof.

66. The composition of claim 64 wherein the vasoactive prostaglandin is prostaglandin E₁ present in the amount of about 0.05 mg to about 0.8 mg.

67. The composition of claim 64 wherein the vasoactive prostaglandin is prostaglandin E₁ present in the amount of about 0.1 mg to about 0.5 mg.

68. The composition of claim 64 wherein the vasoactive prostaglandin is prostaglandin E₁ present in the amount of about 0.2 mg to about 0.3 mg.

69. The composition of claim 64 wherein the piperazinyl quinazoline antihypertensive is present in the amount of about 0.1 mg to about 2.0 mg
